(19) United States
(12) Patent Application Publication
(10) Pub. No.: US 2011/0171284 A1
(43) Pub. Date: Jul. 14, 2011

(54) POVIDONE-IODINE AND SUCROSE WOUND HEALING DRESSING

(76) Inventors: Miles E. Gilman, Miami, FL (US); Joseph S. Bertino, Schenectady, NY (US)

(21) Appl. No.: 12/685,370

(22) Filed: Jan. 11, 2010

(51) Int. Cl. A61L 15/12; A61K 31/74; A61P 17/02

(52) U.S. Cl. 424/445; 424/78.06

(57) ABSTRACT

The present invention relates to a composition and wound dressing for delivering a composition comprising povidone-iodine and sucrose mixture. The composition is gelled and, in one embodiment, is impregnated on a gauze or other like material for application to an exuding or non-exuding wound.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the treatment of wounds by use of a medical dressing comprising povidone-iodine and a sucrose. In particular, where the present invention relates to formulations of povidone iodine and sucrose which have been gelled such that it is no longer liquid and either applied directly to a wound as a pliable product or pre impregnated into a gauze or other medical dressing.

2. Description of Related Art

The use of sucrose solutions made by dissolving sugar in water is known for application as a “wound dressing”. Sucrose based solutions are known to have antibacterial properties as well as are useful for healing of burns and skin ulcers. There is an apparent antibacterial effect from the sucrose and rapid healing frequently can occur in view of the reduced bacterial count from a sucrose solution application. Sucrose is apparently able to stimulate growth of granulation tissue as well. Accordingly, it is not surprising that sucrose solutions, as well as natural liquid products such as maple syrup, sorghum, molasses honey and the like, have been used for hundreds of years.

Povidone-iodine (polyvinylpyrrolidone-iodine complex) is a well known topical anti-infective which is commercially available in several brand name solutions as well as a number of different carriers. Typical formulations include starting from a povidone-iodine power and adding water and other agents to formulate to a desired formulation. Typically, povidone-iodine is applied directly to a wound or to a body area before surgery or a medical procedure to reduce bacterial counts where the possibility of infection is high.

Both sucrose solutions and iodine are typically applied liberally without regard for amount or control of uniformity. The content of previous compositions has not been constant. Even when thicker forms are available, attempts to apply these products generally result in a variable amounts and frequently not enough coverage or concentration of product. Where the product is liquid enough to apply to soak into gauze, it is frequently so fluid that no control is obtained of the product and the product frequently either does not soak into the gauze, soaks in and runs away from the wound, or soaks in and does not leave the gauze to reach the treatment site resulting in haphazard treatment. In addition, the liquid type formulations lead to rapid decomposition of the active ingredients resulting in the need to refrigerate the compositions or prepare compositions immediately before use. Buffers have been added to the compositions to aid in pH based issues with the composition such as stability, however, dosage and uniformity issues have never been sufficiently addressed.

### BRIEF SUMMARY OF THE INVENTION

The present invention relates to the discovery that if povidone iodine and sucrose solution are combined and gelled, they can impregnate a fiber or other type of applied dressing for exposure to the active ingredient, and can be used to give a measured amount of the two ingredients such that their administration remains constant. In addition, in an embodiment where a dosage is formed where the two components are thickened sufficiently such that a pliable mass is formed, then the composition in a fixed dosage can be applied directly to a wound. It can then be covered with a dressing, bandage, or the like. The composition can be buffered but, in the end, the gelling allows for uniformity of the ratio of the composition as well standardization in dosaging.

In one embodiment of the present invention there is a medicinal dressing for the treatment of wound healing comprising:
a) a sucrose;
b) a povidone-iodine solution; and
c) a gelling agent;
   wherein the gelling agent is sufficient to thicken the composition and to substantially prevent the composition from running and release a desired amount of sucrose and povidone to a wound when applied thereto.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings, if any, featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein a “medical dressing” refers to an adjunct used by a person for application to a wound to promote healing and/or prevent further harm, such as by infection. A dressing is designed to be in direct contact with the wound, which makes it different from a bandage, which is primarily used to hold a dressing in place.

A dressing can have a number of purposes, depending on the type, severity and position of the wound, although all purposes are focused towards promoting recovery and preventing further harm to the wound. Key purposes of are dressing are:
Stem bleeding—Helps to seal the wound to expedite the clotting process,
Absorb exudates—Soak up blood, plasma and other fluids exuded from the wound, containing it in one place,
Ease pain—Some dressings may have a pain relieving effect, and others may have a placebo effect,
Debride the wound—The removal of slough and foreign objects from the wound,
Protection from infection and mechanical damage, and
Promote healing—through granulation and epithelialization.

Historically, a medical dressing was usually a piece of material, sometimes cloth, or the like. However, modern dressings include gauzes. Many gauze dressings have a layer of permeable nonstick film over the absorbent gauze to prevent the wound from adhering to the dressing. In the present invention where the composition of the invention is sufficiently gelled to make a pliable composition, the composition itself can be the medical dressing. In the instance where the composition is less thickly gelled it can be used to impregnate gauze or another cloth or fiber typed dressing, or the like, for holding the gel and releasing it or holding it against the wound. Both the gauze and the composition together form the medical dressing. Tape or other means can then be used to hold the medical dressing in place as is typically used for other medical dressings, both medicated and unmedicated.

As used herein “sugar” relates to a non-reducing sugar such as sucrose, granulated sugar or other purified sucrose. By “solution” is meant that the sucrose is dissolved, suspended, or a slurry is created of the sucrose. In one embodiment, a super saturated water solution of sucrose is used as the sucrose. In yet another embodiment there is no water and a granulated sucrose is mixed into the povidone-iodine solution directly.

Povidone-iodine (PVP-I) is a stable chemical complex of polyvinylpyrrolidone (povidone, PVP) and elemental iodine. It contains from 9.0% to 12.0% available iodine, calculated on a dry basis PVP-I, is completely soluble in cold water, ethyl alcohol, isopropyl alcohol, polyethylene glycol, and glycerol. Its stability in solution is much greater than that of tincture of iodine or Lugol's solution. In one embodiment, PVP-I has been formulated at concentrations of 7.5-10.0% in solution. Povidone-iodine stock solution is 10%, comprising 90% water, 8.5% povidone-iodine, 1% available iodine, and 0.5% iodide.

As used herein a “gel” is an agent which when mixed with sufficient water will create a composition with increased viscosity and be able to keep the sucrose positioned for the gelled Povidone iodine solution in a biologically active manner. Selection of a suitable gelling agent involves a number of considerations, including but not limited to, the suitability for the intended medical use, the stability of all the ingredients over time, the compatibility of the gelling agent with the other agents in the composition, as well as the desired properties of the gelling agent including the ability to impregnate a fiber dressing such as gauze. One skilled in the art will be able to choose the desired agent depending on the particular use and intended purpose in view of the present disclosure.

A number of gelling agents are available including various gums, polysaccharides, alginates and various synthetic and natural polymeric compositions. Gelling agents are well known in the art for medical use and based on the criteria set forth herein, a variety of them, as well as newly developed gelling agents, can be used within the present teaching. One embodiment of the gelling agent, the alginates are useful in the present of a polyvalent cation and can be adjusted to a desired consistency in accordance with the principles set out herein. Since they have been used for gelling of other medicinal compositions one skilled in the art would easily be able to incorporate this gelling agent into the present composition. See, for example, U.S. Pat. No. 6,956,144.

Alginates have other advantages in that introduced cations or cations already part of the alginate can be of benefit. For example, calcium containing alginates can be selected where there is bleeding to promote blood clotting.

Other gelling agent could be hydrocolloids and hydrogels. These components can absorb moisture to form a moist healing environment. Consequently, it is possible that they would not be used with a heavily exuding would in which case one would think the alginates would offer better performance. Therefore, one could choose the gelling agent based on the environment of the particular wound being treated. For instance, the hydrocolloids or hydrogels may be incorporated to vary properties such as the amount of fluid absorbed from a wound.

Optional materials can be included in various embodiments of the present invention. These may include various active pharmaceuticals, such as fungicides, additional antibacterials agents, and the like. Fillers could be included, such as calcium carbonate, zinc oxide, barium sulphate, and the like.

A buffer may also be included in the present composition, such as a lactate buffer, a citrate buffer, a phosphate buffer, a potassium hydrogenphthalate buffer, or the like.

The wound dressing may include an optional wound dressing backing, such as gauze. In this particular embodiment, the composition of the present invention can be used to impregnate the fibrous wound dressing for release there from. By impregnating the gauze or other fibrous material, a particular amount of the composition of the present invention can be used to achieve a particular dosage for administration to the wound.

No particular limitation is imposed on the production method for the preparation of the gelled composition of the present invention. The manufacturing process can be a manual or automated process as desired. For example, an aqueous standard povidone-iodine solution can be used and the sugar, gelling agent and the like dissolved in the PVP solution. Other methods could be to dissolve each ingredient in its own aqueous solution and then mix the ingredients to achieve the gelled composition. Additional ingredients can be added in a like manner. In other embodiments, the ingredient, the dressing, and the like can be assembled automatically by machine. Such manufacturing is within the skill in the art in view of the present disclosure.

A more complete understanding can be obtained from the examples that follow but these are for the purposes of illustration only and not intended to be limiting unless otherwise specified.

### EXAMPLE 1

### Composition for Exuding Wounds

In this embodiment 30 ml of a standard 10% PVP solution (Betadine) is mixed with 30 grams of granulated table sugar (sucrose) until the sugar is dissolved. No heat is applied, but mild agitation increases the dissolution rate. Once the sugar is in solution, 5 g of sodium alginate is added and stirred until a consistent gel is formed. The alginate absorbs water from the solution to form the gel. The gel is then applied to a piece of inert material, such as wax paper or saran wrap, as a base. A piece of sterile gauze sheeting is placed on the base and then the gelled mixture is applied evenly on the gauze using a spatula until evenly distributed. A piece of the base material is then also placed on top of the gel/gauze and a roller is run over the surface to evenly distribute the gel throughout the gauze for penetration throughout the gauze. The base material can be removed and the gauze cut, if necessary to the proper size. The gauze, if desired, can be attached to a sterile adhesive bandage for use on a particular size wound.

### EXAMPLE 2

### Composition for Non-Exuding Wounds

The composition and gauze impregnation of Example 1 is utilized, however, only 2.5 g of sodium alginate is used in making the gelled composition.

It will be apparent to those having ordinary skill in the art that many changes and modifications can be made without departing from the spirit and scope of the invention as set forth herein in the claims which follow.

What is claimed is:

1. In one embodiment of the present invention there is a medical dressing for the treatment of wound healing comprising:
a) sucrose;
b) a povidone-iodine solution; and
c) a gelling agent;
wherein the gelling agent is sufficient to thicken the composition and to substantially prevent the composition from running and release a desired amount of sucrose and povidone to a wound when applied thereto.

2. A medicinal dressing according to claim 1 wherein the sugar/povidone-iodine is present in a ratio of from about 3 to 1 to about 4 to 1.

3. A medicinal dressing according to claim 1 wherein the dressing is impregnated in a reinforcing fiber dressing for application to a wound and release therefrom.

4. A medicinal dressing according to claim 2 wherein the dressing is medical gauze.

5. A medicinal dressing according to claim 1 wherein the dressing is moldable to fit a wound without use of an additional dressing.

6. A medicinal dressing according to claim 1 wherein the mixture comprises 70 parts of sugar to 30 parts of povidone-iodine.

7. A medicinal dressing according to claim 1 wherein the composition is formulated for application to a single wound.

8. A medicinal dressing according to claim 1 wherein the gelling agent is sodium alginate.

9. A method of treating a wound comprising the application of the composition of claim 5 to the wound.
